(19) United States
(12) Patent Application Publication
(10) Pub. No.: US 2006/0004312 A1
(43) Pub. Date: Jan. 5, 2006

(54) Joint mobilizing tool

(76) Inventors: Alisa Renee Stoltz, Indianapolis, IN (US); Andrew Joseph Stoltz, Indianapolis, IN (US)

Correspondence Address: Alisa Renee Stoltz, 5953 N. Compton St., Indianapolis, IN, 46220, US

(21) Appl. No.: 11/167,325

(22) Filed: Jun. 27, 2005

Related U.S. Application Data
(60) Provisional application No. 60/583,383, filed on Jun. 28, 2004.

(51) Int. Cl. A61F 5/00

(52) U.S. Cl. 602/5; 602/21; 602/20

(57) ABSTRACT

There is disclosed a joint mobilizing tool for mobilizing a human joint. A method of using the joint mobilizing tool is also disclosed. This product's purpose is to allow the person performing a joint mobilization to keep their wrist in a more neutral position instead of extending or deviating their wrist. An extended or deviated wrist is much more prone to injury than a wrist in a neutral position.

This application claims benefit under 35 U.S.C & 119 (e) of U.S. provisional patent application No. 60/583383 which was filed on Jun. 28, 2004 and is hereby incorporated for reference herein.

### BACKGROUND

This application relates to the mobilizing of human joints

### SUMMARY

According to the patent disclosure, a joint mobilizing tool is provided for assistance with the mobilization of the human joint.

### BRIEF DESCRIPTION OF DRAWINGS

FIG. 1 is the tool comprising of a pad, a mount and a handle. The pad is adapted to the mount. The mount is adapted to the c-shaped handle that has finger receiving notches.

The mount and handle will be comprised of one solid piece.

FIG. 2 is a T shaped handle with finger receiving notches that can be manufactured to connect to the mount, in place of the c-shaped handle, if the client prefers.

### DETAILED DESCRIPTION OF DRAWINGS

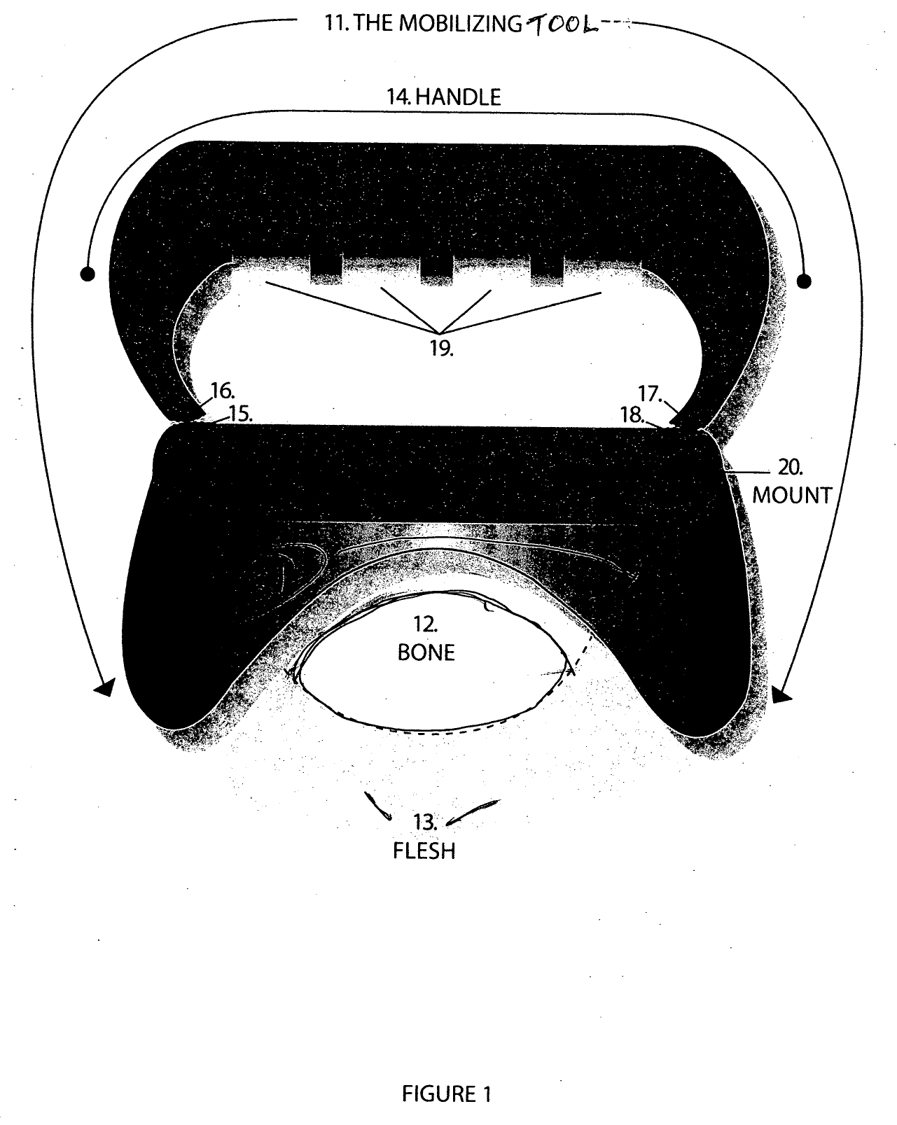

Referring to FIG. 1, there is shown a joint mobilizing tool 11 for use in mobilizing a human joint by contact with the human bone 12 associated with human flesh around human bone 13.

The joint mobilizing tool can be used with a variety of joints.

Referring to FIG. 1, there is a c-shaped handle 14 for use to be gripped.

Referring to FIG. 1, the handle 14 is connected to the 1^{st} end of the mount 15 to the 1^{st} end of the handle 16.

The 2^{nd} end of the handle 17 is connected to the second end of the mount 18.

The c-shaped handle comprises four finger receiving notches 19.

The mount 20 attaches to the pad 21.

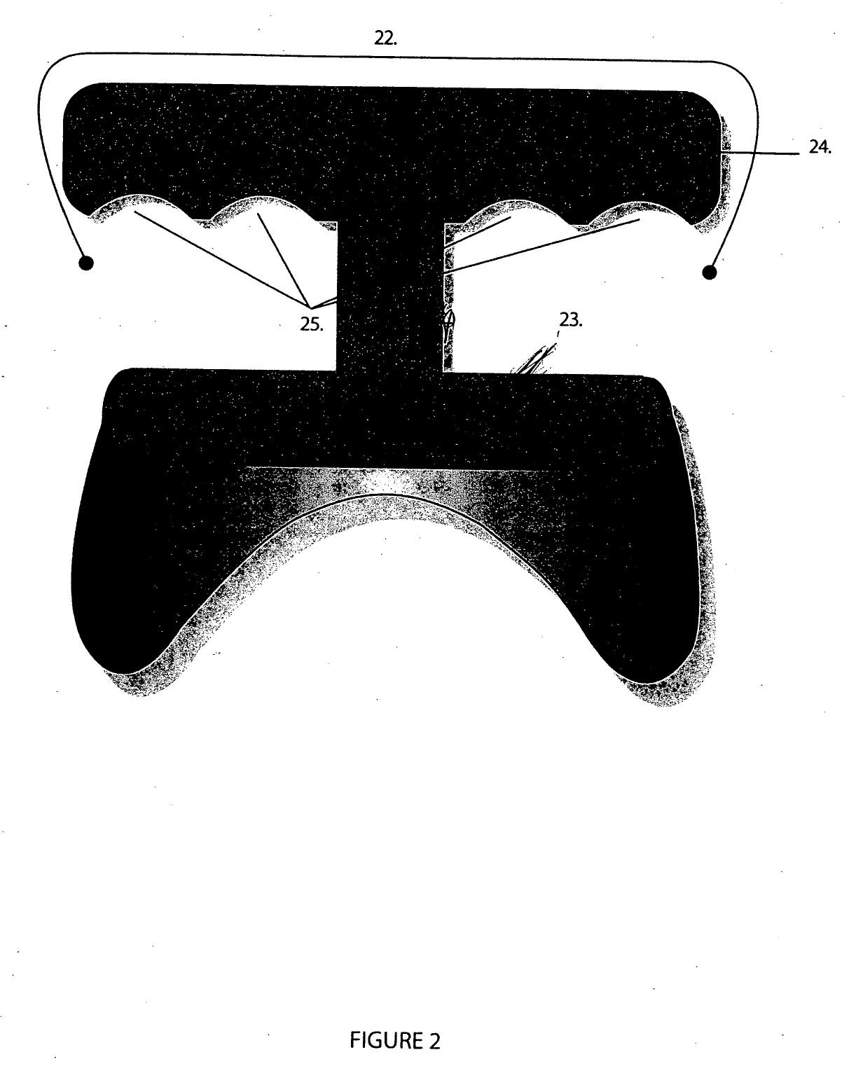

Referring to FIG. 2 there is shown a T-shaped handle 22.

Referring to FIG. 2 there is shown the end of the T-shaped handle connected to the middle of the mount 23.

The body of the T-shaped handle 24 comprises 4 finger-receiving notches 25.

Referring to FIG. 1, the C-shaped handle 14 and mount 20 will be made of plastic.

Referring to FIG. 2, the T-shaped handle 22, will be made of plastic.

Referring to FIGS. 1 and 2 both the C-shaped handle 14 and T-shaped handle 22 allow the wrist of the person grasping the handle 14 and 22 to be in a neutral position.

1. A joint mobilizing tool for mobilizing a human joint, comprising: a pad adapted to conform to a bone associated with the human joint, a mount to which the pad is secured, and a handle secured to the mount and adapted to be gripped by a hand of a person so as to allow a wrist of the person associated with the hand to remain in a neutral position during mobilization of the human joint.

2. The joint mobilizing tool of claim 1, wherein the handle is C-shaped.

3. The joint mobilizing tool of claim 1, wherein the handle comprises a body adapted to be gripped by the hand, a first connector connecting a first end of the handle and a first end of the mount, and a second connector connecting a second end of the handle and a second end of the mount to promote distribution of a load applied to the body across the pad.

4. The joint mobilizing tool of claim 1, wherein the handle is T-shaped.

5. The joint mobilizing tool of claim 4, wherein the handle comprises a body adapted to be gripped to the handle and a shaft connecting a middle portion of the body to the mount.

6. The joint mobilizing tool of claim 1, wherein the handle comprises finger-receiving notches.

7. The joint mobilizing tool of claim 1, wherein the pad comprises foam adapted to conform to the shape of the bone.

8. The joint mobilizing tool of claim 1, wherein the pad comprises a bone-receiving notch in a relaxed state of the pad.

9. A method of mobilizing a human joint, the method comprising the acts of: applying a force to the human joint with a joint mobilizing aid such that a wrist of a person applying the force remains in a more neutral position.

10. The method of claim 9, wherein the applying act comprises applying the force through a handle gripped by the person and through a pad secured to the handle and in contact with a bone of the human joint.
